(19) United States
(12) Patent Application Publication
(10) Pub. No.: US 2025/0073013 A1
(43) Pub. Date: Mar. 6, 2025

(54) SYSTEMS AND METHODS FOR LIP IMPLANTS

(71) Applicant: Caitlin Iorio, Salt Lake City, UT (US)

(72) Inventor: Caitlin Iorio, Salt Lake City, UT (US)

(21) Appl. No.: 18/823,467

(22) Filed: Sep. 3, 2024

Related U.S. Application Data
(60) Provisional application No. 63/535,992, filed on Aug. 31, 2023.

(51) Int. Cl. A61F 2/00

(52) U.S. Cl. CPC A61F 2/0059

(57) ABSTRACT

A lip implant for implantation into lips of a patient is provided. The lip implant includes an upper lip implant and a lower lip implant. The upper lip implant and the lower lip implant are formed of an acrylate hydrogel.

### CROSS REFERENCE TO RELATED APPLICATION

This application claims priority to and the benefit of U.S. Provisional Patent Application No. 63/535,992, filed Aug. 31, 2023, and entitled SYSTEMS AND METHODS FOR LIP IMPLANTS, the disclosure of which is incorporated herein by reference in its entirety.

### TECHNICAL FIELD

The present disclosure relates generally to systems and methods for lip implants and for methods of designing and manufacturing the lip implants.

### BACKGROUND OF CERTAIN ASPECTS OF THE DISCLOSURE

Lip augmentation is used to add volume to the lips of a patient, creating a more youthful appearance. Some patients may elect to undergo lip augmentation because their lips may be thinning or aging in an undesirable manner. There are several types of lip augmentation, including lip implants, lip fillers, fat transfers, and lip lifts. Most lip augmentation only temporarily adds volume to the lips of the patient. For example, lip fillers only last for a relatively short period of time (2-5 months) before additional lip filler needs to be injected. As such, lip fillers are merely a temporary solution.

Lip implants last for as long as the implant is implanted into the patient's lips. Specifically, lip implants include a sack of material implanted into the patient's lips to increase the volume and/or aesthetics of the patient's lips and face. However, at least some known lip implants are hard and uncomfortable for the patient and/or may contort the patient's lips and/or face in undesirable ways.

Accordingly, there is a need for a system and method that designs, manufactures, and implants lip implants that are softer and more aesthetically pleasing than existing lip augmentation solutions.

### BRIEF SUMMARY OF SOME ASPECTS OF THE DISCLOSURE

One aspect of the present disclosure relates to a lip implant for implantation into lips of a patient. The lip implant includes an upper lip implant and a lower lip implant. The upper lip implant and the lower lip implant are formed of an acrylate hydrogel.

Another aspect of the present disclosure relates to a method of designing lip implants. The method includes scanning at least a lip of a patient using a scanner to generate anatomical data of the patient. The method also includes transmitting the anatomical data to a computing device. The method further includes creating a three dimensional model of the patient's lips using the computing device. The method also includes designing the lip implant based at least partially on the three dimensional model.

Another aspect of the present disclosure relates to a method of manufacturing lip implants. The method includes depositing a first layer of acrylate hydrogel on a platform of an additive manufacturing system. The method also includes curing the first layer of acrylate hydrogel using a UV light of the additive manufacturing system. The method further includes depositing a second layer of acrylate hydrogel on the first layer. The method also includes curing the second layer of acrylate hydrogel using the UV light of the additive manufacturing system.

There are other novel aspects and features of this disclosure. They will become apparent as this specification proceeds. Accordingly, this brief summary is provided to introduce a selection of concepts in a simplified form that are further described below in the detailed description. The summary and the background are not intended to identify key concepts or essential aspects of the disclosed subject matter, nor should they be used to constrict or limit the scope of the claims. For example, the scope of the claims should not be limited based on whether the recited subject matter includes any or all aspects noted in the summary and/or addresses any of the issues noted in the background.

### BRIEF DESCRIPTION OF THE DRAWINGS

A further understanding of the nature and advantages of the embodiments may be realized by reference to the following drawings. In the appended figures, similar components or features may have the same reference label.

FIG. 1 illustrates two views of lip implants described herein not implanted into a patient's lips and three views of the process of implanting the lip implants into a patient's lips in accordance with aspects of the present disclosure.

FIG. 2 illustrates a front view of the lips of the patient in accordance with aspects of the present disclosure.

FIG. 3 illustrates a schematic front view of the lips of the patient in accordance with aspects of the present disclosure.

FIG. 4 illustrates a schematic side view of a face of the patient including the lips of the patient in accordance with aspects of the present disclosure.

FIG. 5 illustrates a schematic side view of a head of the patient including the lips of the patient in accordance with aspects of the present disclosure.

FIG. 6 illustrates another front view of the lips of the patient in accordance with aspects of the present disclosure.

FIG. 7 illustrates a flow chart of a method for designing lip implants in accordance with aspects of the present disclosure.

FIG. 8 illustrates a perspective view of an additive manufacturing system configured to manufacture the lip implants described herein in accordance with aspects of the present disclosure.

FIG. 9 illustrates a perspective view of the print head illustrated in FIG. 8 in accordance with aspects of the present disclosure.

FIG. 10 illustrates a flow chart of a method of manufacturing lip implants in accordance with aspects of the present disclosure.

While the embodiments described herein are susceptible to various modifications and alternative forms, specific embodiments have been shown by way of example in the drawings and will be described in detail herein. However, the exemplary embodiments described herein are not intended to be limited to the particular forms disclosed. Rather, the instant disclosure covers all modifications, equivalents, and alternatives falling within the scope of the appended claims.

### DETAILED DESCRIPTION

The systems and methods disclosed herein relate to lip implants and methods for designing and manufacturing lip implants. As discussed above, at least some known lip implants are hard and uncomfortable for a patient. To correct these deficiencies, the lip implants described herein are manufactured or formed from a softer, more comfortable material that improves the overall comfort of the patient and enables a medical professional to tailor the lip implant to the patient, improving the results or aesthetics of the patient after the lip implant has been implanted into the patient. Moreover, the methods described herein enable a medical practitioner to customize and design the lip implants described herein to improve comfort of the lip implants.

Lip augmentation is used to add volume to the lips of the patient, creating a more youthful appearance, because the lips may be thinning or aging in an undesirable manner. There are several types of lip augmentation, including lip implants, lip fillers, fat transfers, and lip lifts. Most lip augmentation only temporarily adds volume to the lips of the patient. For example, lip fillers include injections that only last for a relatively short period of time (2-5 months) before additional lip filler needs to be injected. However, lip implants last for as long as the implant is implanted into the patient's lips.

As described above, at least some known lip implants are formed of a solid material that is non-degradable. Specifically, at least some known lip implants are formed of silicone formed into a predetermined, generic shape. At least some known lip implants are formed of a relatively hard silicone formed into a relatively cylindrical shape that is not ergonomic. More specifically, the lip implant may have a hardness rating of 0 to 50 as measured by the durometer A-scale rating.

In contrast, the lip implants described herein are formed of an acrylate hydrogel that, when cured, is significantly softer than the currently used silicone lip implants. More specifically, the lip implants described herein are formed of polyacrylic hydrogels and may also include acrylic acid and acrylamide. Acrylic acid is a monomer that is cross-linked to produce hydrogels with high water absorbing capacity than a single or multicomponent system. Acrylic acid is also known as propenoic acid and has a carboxylic acid group with a carboxylic end that is connected to a vinyl group. The carboxylic acid increases the ionic strength and sensitivity to pH of the hydrogel. Acrylamide hydrogels exhibit volume transition to physical and chemical stimuli. Acrylamide hydrogels have sufficient amount of hydrolytic stability which can be further improved by introducing acrylamides with groups such as alkyl and hydroxyl alkyl. Additionally, the lip implants described herein may be formed of a stiffness-tunable hydrogel that may be formed by changing the crosslinking degree of gels, stimulating external conditions such as UV light, changing the molecular weight of the material, and adding different nanomaterials. The polyacrylic hydrogels are softer and more comfortable for the patient because the polyacrylic hydrogels: (1) have a storage modulus G′ of less than 2500 Pa and tan delta (TD) of less than 0.2 in extrusion-based printing, (2) are capable of absorbing or diffusing biological fluids when in the body, and (3) may be porous to biological fluids. As such, the acrylate hydrogel lip implants described herein are significantly softer and more comfortable than the currently used silicone lip implants.

The acrylate hydrogels described herein include a non-degradable acrylate hydrogel ink that is deposited with a UV print-and cure printhead. This will provide a proper storage modulus G′ values that will allow flexibility and ability to flex with the movement of the lips. The storage modulus represents the elastic portion of the viscoelastic behavior and describes how a material is able to retain its shape when force is applied. The material will be softer than the current silicone implants. The acrylate hydrogel will also have higher water-absorbing properties. Hydrogels can be derived from natural polymers, synthetic polymers, or a combination of both. Hydrogels made of synthetic polymers generally demonstrate superior properties compared to hydrogels derived from natural polymers. These synthetic polymer hydrogels have long life-to-failure duration, great water absorption capacity, and improved mechanical properties such that the structure can be tailored to its functionality. Superabsorbent hydrogels based on acrylic acid and acrylamide are typically used for various applications in biomedical and tissue engineering.

Furthermore, the design process is configured to increase the comfort of the lip implant for the patient. More specifically, in the methods and design processes described herein, a medical professional uses a scanner to scan the anatomy of the patient. The scanner may include a three dimensional scanner that is capable of scanning both the inside of the lips/mouth and the outside of the lips/mouth. The scanner sends data to a computing device and the computing device creates a three dimensional model of the patient's lips, mouth, and face.

After the three dimensional model has been created, a medical professional analyzes the three dimensional model and designs the lip implant for comfort based on the three dimensional model. First, the medical professional evaluates the patient's anatomy and ethnicity and compares this to the patient's desired outcomes. Next, the medical professional designs the lip implant based on the patient's desired outcomes and generally accepted aesthetic conventions. For example, based on the study of ideal proportions beginning with Leonardo Da Vinci and the golden ratio, the medical professional takes into account the accepted norms of the ideal aesthetic proportions of the lips. The ideal proportional height of the upper lip to lower lip ratio is 1:1.6 or generally accepted as 1:2. The anatomical landmarks of the lips vary based on ethnicity vary but, the lips generally include multiple subunits including the subnasale, the cupid's bow, the philtral columns, the philtral dimple, the melolabial fold, labiomental crease, the upper lip tubercles, the lower lip tubercles, and the vermillion border.

Horizontal lip position and fullness may be determined by two separate methods. The first method is the line method where a line is drawn from the patient's subnasale (Sn) through the labrale inferius (Li) to the pogonion (Pg). Upper and lower lip should lie 3.5 and 2.2 mm anterior to this line, respectively. The second method is the nasomental angle method where a first line is drawn along the bridge of the patient's nose and a second line is drawn from the tip of the patient's nose to the patient's chin. An angle is formed where the first and second lines intersect that is about 1200 to about 132°. The lips should fall just behind the second line at a distance of 4 mm for the upper lip and 2 mm for the lower lip.

The mucosal or “red” lips have identifiable landmarks that indicate aesthetically pleasing volume. These include three the tubercles of the upper lip and the two tubercles of the lower lip which interdigitate. These tubercles are augmented by the lip implants described herein to add to the volume of the upper and lower lips according to the description provided above. Because the tubercles vary in shape and size between patient's, generic and mass produced cylindrical lip implants fail to produce consistently aesthetically pleasing results and the lip implants described herein are customized to a patient's anatomy and desires to achieve consistently aesthetically pleasing results.

After medical professional designs the lip implant, the custom designed lip implant is manufactured using an additive, or extrusion-based manufacturing system. The additive manufacturing systems described herein enable the lip implants to be individually manufactured to the medical professional's specifications. Specifically, the additive manufacturing systems described herein include a three dimensional printing system that manufactures the lip implants by depositing or printing the acrylate hydrogel material in layers such that the lip implant is formed layer by layer. Additionally, because acrylate hydrogels are slightly viscous materials when printed, the three dimensional printing system includes an ultra-violet light (UV light) that cures the acrylate hydrogel in the printed shape as the lip implant is printed. As such, the three dimensional printing system described herein enables the medical professional to custom design a lip implant based on the anatomy of the patient, individually manufacture the lip implant, and cure the lip implant in the designed shape. Additionally, the three dimensional printing systems described herein are capable of forming the lip implants in softer materials. Accordingly, the three dimensional printing systems described herein enable the medical professional to custom design and manufacture softer lip implants that improve the overall comfort and aesthetic appearance of the patient.

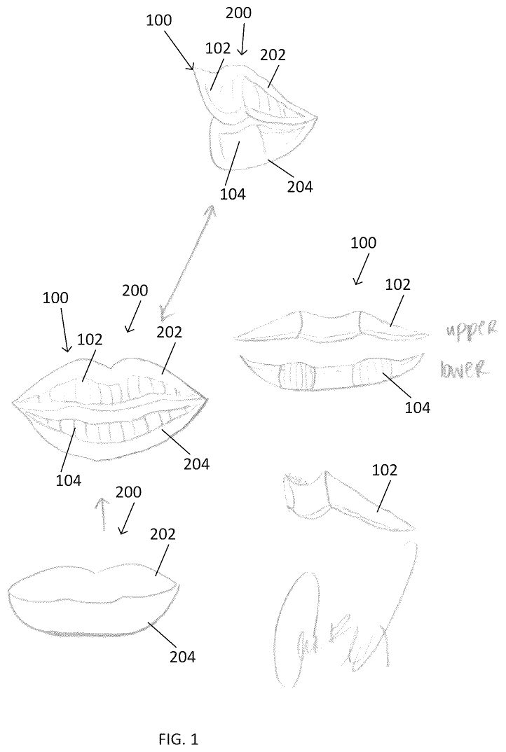

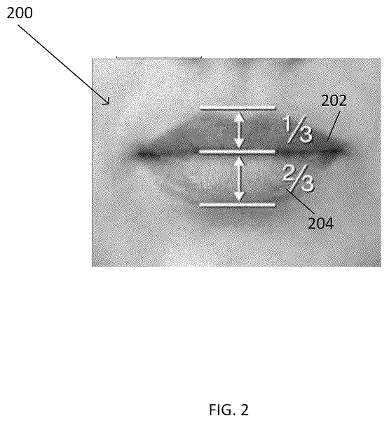

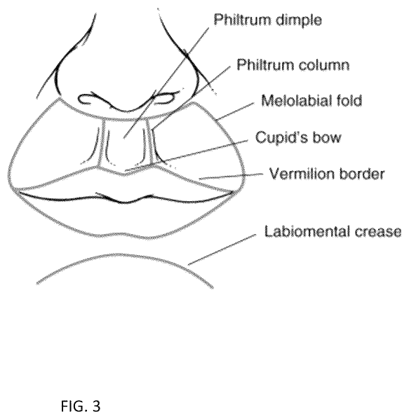

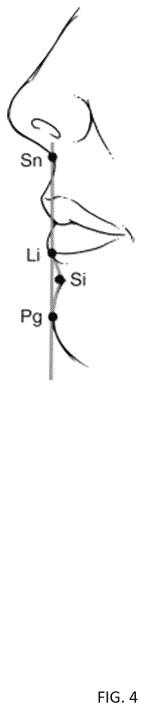

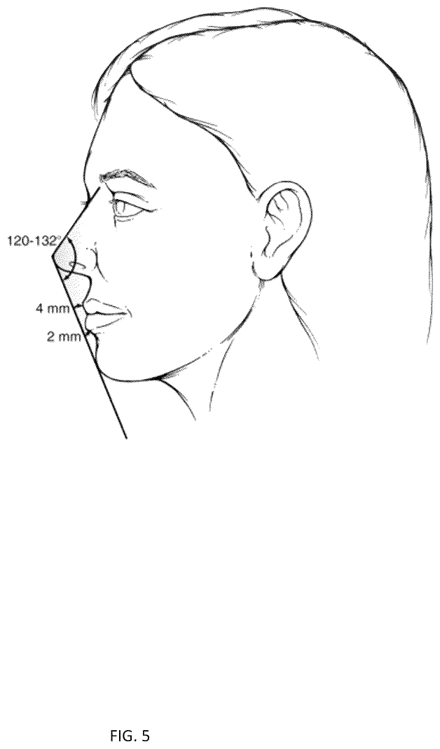

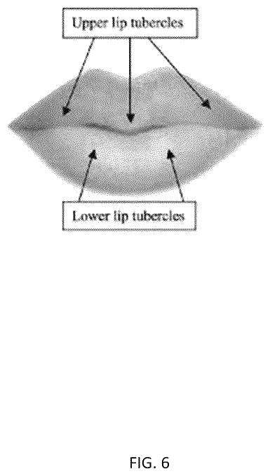

FIG. 1 illustrates two views of lip implants 100 described herein not implanted into a patient's lips and three views of the process of implanting the lip implants 100 into a patient's lips 200. As shown in FIG. 1, lip implants 100 include an upper lip implant 102 and a lower lip implant 104 corresponding to an upper lip 202 and a lower lip 204 of the patient. FIG. 2 illustrates a front view of the lips 200 of the patient. FIG. 3 illustrates a schematic front view of the lips 200 of the patient. FIG. 4 illustrates a schematic side view of a face of the patient including the lips 200 of the patient. FIG. 5 illustrates a schematic side view of a head of the patient including the lips 200 of the patient. FIG. 6 illustrates another front view of the lips 200 of the patient.

As shown in FIGS. 2-6, the lips 200 of the patient generally include the subnasale, the cupid's bow, the philtral columns, the philtral dimple, the melolabial fold, labiomental crease, the upper lip tubercles, the lower lip tubercles, and the vermillion border. Similarly, the lip implants 100 also generally include the subnasale, the cupid's bow, the philtral columns, the philtral dimple, the melolabial fold, labiomental crease, the upper lip tubercles, the lower lip tubercles, and the vermillion border. However, portions of the subnasale, the cupid's bow, the philtral columns, the philtral dimple, the melolabial fold, labiomental crease, the upper lip tubercles, the lower lip tubercles, and the vermillion border of the lip implants 100 described herein may be increased or decreased in relative size to augment specific areas of the lips 200 of the patient.

The lip implants 100 described herein are formed of an acrylate hydrogel that, when cured, is significantly softer than the currently used silicone lip implants. More specifically, the lip implants 100 described herein are formed of polyacrylic hydrogels and may also include acrylic acid and acrylamide. Acrylic acid is a monomer that is cross-linked to produce hydrogels with high water absorbing capacity than a single or multicomponent system. Acrylic acid is also known as propenoic acid and has a carboxylic acid group with a carboxylic end that is connected to a vinyl group. The carboxylic acid increases the ionic strength and sensitivity to pH of the hydrogel. Acrylamide hydrogels exhibit volume transition to physical and chemical stimuli. Acrylamide hydrogels have sufficient amount of hydrolytic stability which can be further improved by introducing acrylamides with groups such as alkyl and hydroxyl alkyl. Additionally, the lip implants described herein may be formed of a stiffness-tunable hydrogel that may be formed by changing the crosslinking degree of gels, stimulating external conditions such as UV light, changing the molecular weight of the material, and adding different nanomaterials. The polyacrylic hydrogels are softer and more comfortable for the patient because the polyacrylic hydrogels: (1) have a storage modulus G′ of less than 2500 Pa and tan delta (TD) of less than 0.2 in extrusion-based printing, (2) are capable of absorbing or diffusing biological fluids when in the body, and (3) may be porous to biological fluids. As such, the acrylate hydrogel lip implants 100 described herein are significantly softer and more comfortable than the currently used silicone lip implants.

The acrylate hydrogels described herein include a non-degradable acrylate hydrogel ink that is deposited with a UV print-and cure printhead. This will provide a proper storage modulus G′ values that will allow flexibility and ability to flex with the movement of the lips. The storage modulus represents the elastic portion of the viscoelastic behavior and describes how a material is able to retain its shape when force is applied. The material will be softer than the current silicone implants. The acrylate hydrogel will also have higher water-absorbing properties. Hydrogels can be derived from natural polymers, synthetic polymers, or a combination of both. Hydrogels made of synthetic polymers generally demonstrate superior properties compared to hydrogels derived from natural polymers. These synthetic polymer hydrogels have long life-to-failure duration, great water absorption capacity, and improved mechanical properties such that the structure can be tailored to its functionality. Superabsorbent hydrogels based on acrylic acid and acrylamide are typically used for various applications in biomedical and tissue engineering.

Specifically, as described in methods described herein, a medical professional may determine that a certain aspect of the patient's lips 200 may need augmentation while other aspects of the patient's lips 200 do not need augmentation. For example, the medical professional may determine that the lower lip 204 of the patient needs a large amount of augmentation but the upper lip 202 of the patient only needs a small amount of augmentation. As such, the medical professional may design the upper lip implant 102 to be relatively small compared to the lower lip implant 104 in order to achieve the ideal proportional height of the upper lip to lower lip of approximately 1:2, 1:1.25 for patients of Chinese descent, 1:1.1 for patients of Korean descent, 1:1.43 for patients of Caucasian descent, or ideally 1:1.6 based on the golden ratio.

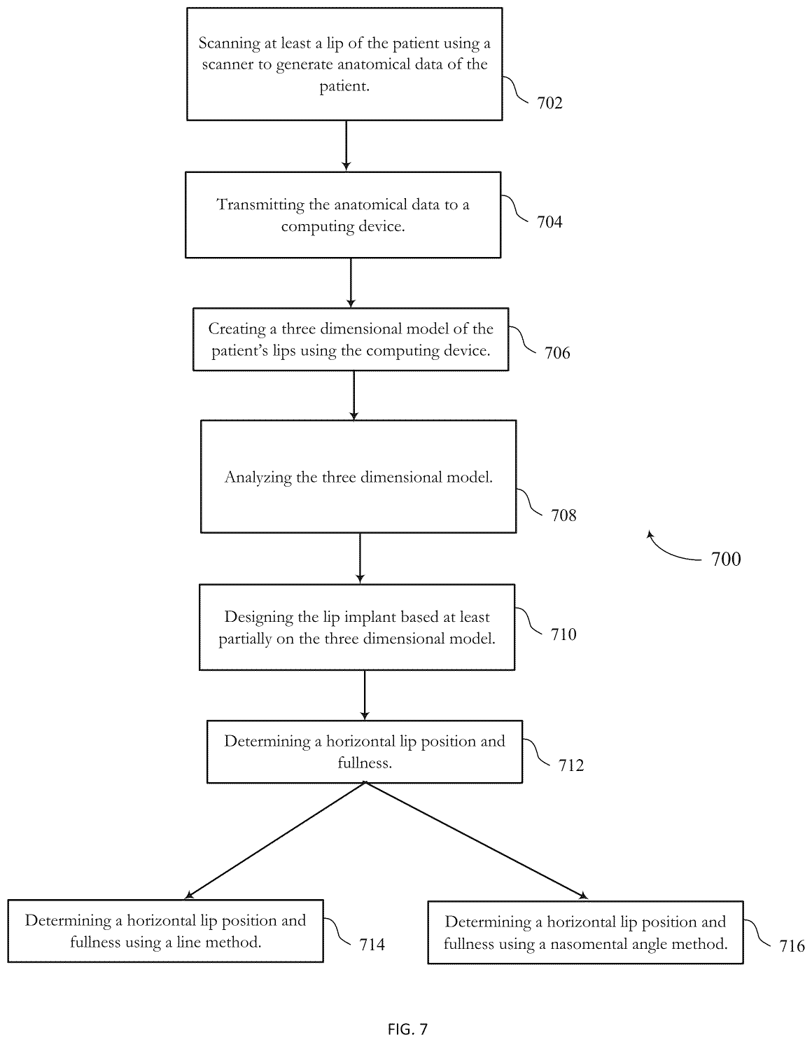

FIG. 7 illustrates a flow chart of a method 700 for designing lip implants 100 in accordance with aspects of the present disclosure. The operations of method 700 may be implemented by a device or its components as described herein. For example, the operation of method 700 may be performed by a computing device or with the assistance of a computing as described herein. The computing device may operate to perform some or all of the functions associated with the systems described herein. In some examples, the computing device may execute a set of instructions to perform the functions or steps described herein. Additionally, or alternatively, the computing device may perform aspects of the functions described herein using special purpose hardware.

The method 700 includes scanning 702 at least a lip of the patient using a scanner to generate anatomical data of the patient. More specifically, a medical professional uses a scanner to scan the anatomy of the patient. The scanner may include a three dimensional scanner that is capable of scanning both the inside of the lips/mouth and the outside of the lips/mouth. The method 700 also includes transmitting 704 the anatomical data to a computing device. The scanner sends the anatomical data to a computing device using any data transmission method. The method also includes creating 706 a three dimensional model of the patient's lips using the computing device.

The method 700 further includes analyzing 708 the three dimensional model. The method 700 also includes designing 710 the lip implant based at least partially on the three dimensional model. First, the medical professional evaluates the patient's anatomy and ethnicity and compares this to the patient's desired outcomes. Next, the medical professional designs the lip implant based on the patient's desired outcomes and generally accepted aesthetic conventions. For example, based on the study of ideal proportions beginning with Leonardo Da Vinci and the golden ratio, the medical professional takes into account the accepted norms of the ideal aesthetic proportions of the lips. The ideal proportional height of the upper lip to lower lip ratio is 1:1.6 or generally accepted as 1:2. The anatomical landmarks of the lips vary based on ethnicity vary but, the lips generally include multiple subunits including the subnasale, the cupid's bow, the philtral columns, the philtral dimple, the melolabial fold, labiomental crease, the upper lip tubercles, the lower lip tubercles, and the vermillion border.

Designing 710 the lip implant based at least partially on the three dimensional model typically includes determining 712 a horizontal lip position and fullness. In some embodiments, determining 712 a horizontal lip position and fullness includes determining 714 a horizontal lip position and fullness using a line method. The line method includes drawing or rendering a line (illustrated in FIG. 4) from the patient's subnasale (Sn) through the labrale inferius (Li) to the pogonion (Pg). Upper and lower lip should lie 3.5 and 2.2 mm anterior to this line, respectively. In some embodiments, determining 712 a horizontal lip position and fullness includes determining 716 a horizontal lip position and fullness using a nasomental angle method. The nasomental angle method includes drawing or rendering a first line along the bridge of the patient's nose and a second line from the tip of the patient's nose to the patient's chin. An angle is formed where the first and second lines intersect that is about 1200 to about 132°. The lips should fall just behind the second line at a distance of 4 mm for the upper lip and 2 mm for the lower lip.

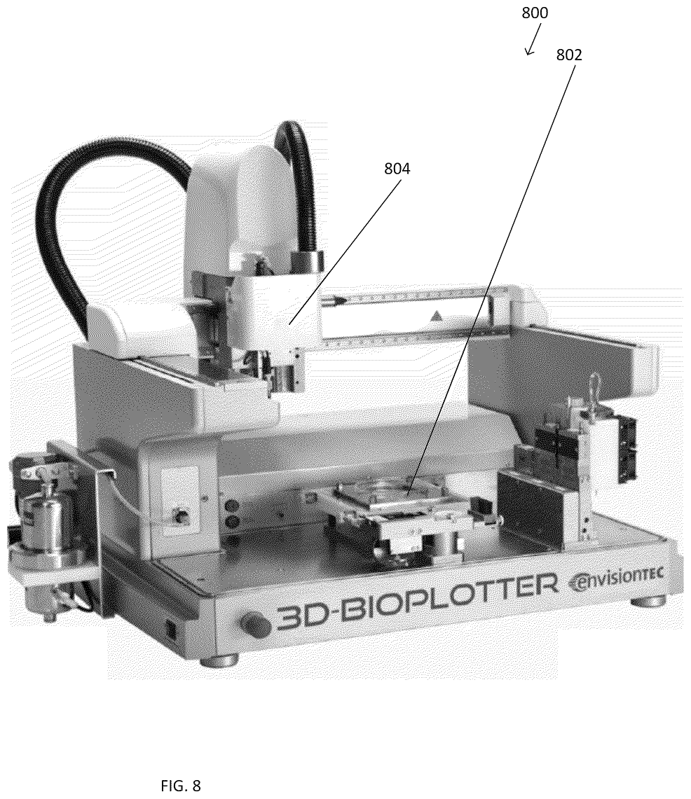

FIG. 8 illustrates a perspective view of an additive manufacturing system 800 configured to manufacture the lip implants 100 described herein. The additive manufacturing system 800 described herein enables the lip implants 100 to be individually manufactured to the medical professional's specifications. Specifically, the additive manufacturing systems 800 described herein include a three dimensional printing system that manufactures the lip implants 100 by depositing or printing the acrylate hydrogel material in layers such that the lip implant 100 is formed layer by layer. Additionally, because acrylate hydrogels are slightly viscous materials when printed, the three dimensional printing system includes an ultra-violet light (UV light) that cures the acrylate hydrogel in the printed shape as the lip implant 100 is printed. As such, the three dimensional printing system described herein enables the medical professional to custom design a lip implant based on the anatomy of the patient, individually manufacture the lip implant, and cure the lip implant in the designed shape. Additionally, the three dimensional printing systems described herein are capable of forming the lip implants 100 in softer materials. Accordingly, the three dimensional printing systems described herein enable the medical professional to custom design and manufacture softer lip implants 100 that improve the overall comfort and aesthetic appearance of the patient.

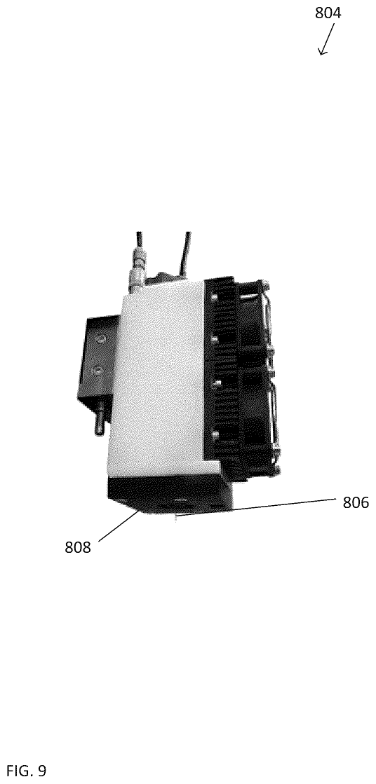

As shown in FIG. 8, the additive manufacturing system 800 includes a platform 802 and a print head 804. FIG. 9 illustrates a perspective view of the print head 804. The print head 804 includes a UV print-and cure printhead including a needle 806 and a UV light 808 positioned proximate the needle 806. During operations, the acrylate hydrogel is pressurized such that the acrylate hydrogel flows out to the needle 806 onto the platform 802. The acrylate hydrogel is typically fairly viscous such that it remains stationary for a short period of time when not pressurized. That is, the viscosity of unpressurized acrylate hydrogel enables the acrylate hydrogel to maintain the shape it is printed in by the needle 806 for a short period of time. Additionally, pressurizing the acrylate hydrogel enables the acrylate hydrogel to flow and be shaped layer-by-layer by the needle 806.

After the acrylate hydrogel has been deposited on the platform 802, the UV light 808 is directed toward the acrylate hydrogel to cure the acrylate hydrogel in place. Acrylate hydrogels are cured by polymerizing or crosslinking the polymer chains in the acrylate hydrogel. Crosslinking is the process of joining two or more polymer chains by chemical and/or physical bonding. The UV light 808 increases the temperature of the acrylate hydrogel causing the polymer chains within the acrylate hydrogel to polymerize or crosslink. As such, the UV light 808 causes the acrylate hydrogel to harden in the printed shaped, forming the lip implants 100 described herein.

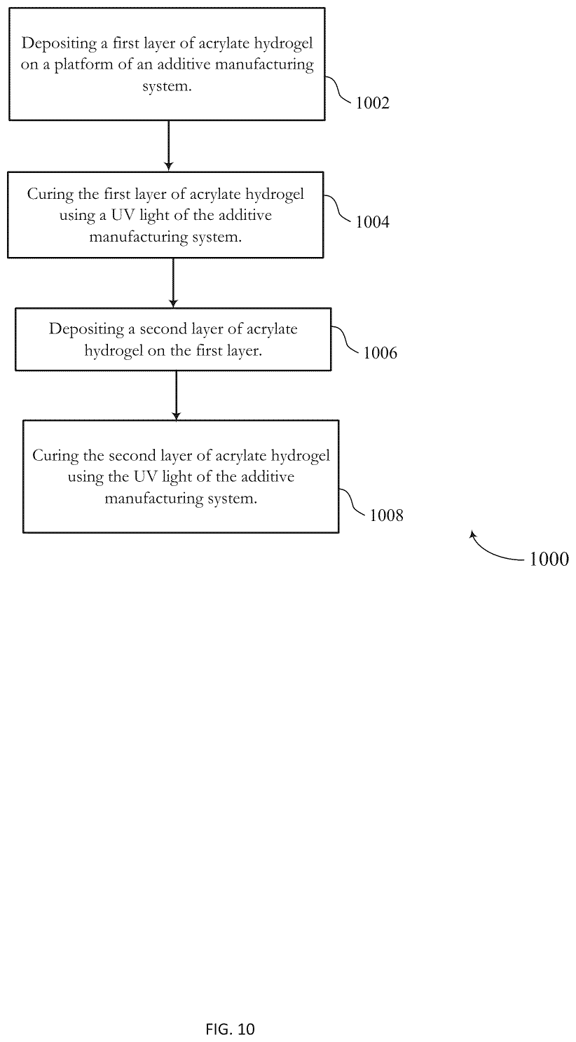

FIG. 10 illustrates a flow chart of a method 1000 of manufacturing lip implants 100 in accordance with aspects of the present disclosure. The method 1000 includes depositing 1002 a first layer of acrylate hydrogel on a platform of an additive manufacturing system. The method 1000 also includes curing 1004 the first layer of acrylate hydrogel using a UV light of the additive manufacturing system. The method 1000 includes depositing 1006 a second layer of acrylate hydrogel on the first layer. The method 1000 also includes curing 1008 the second layer of acrylate hydrogel using the UV light of the additive manufacturing system.

After the lip implants 100 have been manufacture, the medical professional implants the lip implants 100 in the patient's lips 200. The methods used to implant the lip implants 100 include surgically implanting the lip implants 100 by creating an incision on either corner of mouth, or the oral commissure, of the patient, inserting a clamp into the incision, creating a pocket in the lips of the patient 200 configured to receive the lip implants 100, opening the pocket with the clamp, and inserting the lip implants 100 into the pocket. A central incision may also be utilized in the mucosa, or red lip, to further assist in implanting the lip implants 100. In alternative embodiments, the lip implants 100 may be implanted into the lips 200 of the patient using any method that enables the systems and methods described herein to operate as described herein.

It should be noted that the methods described herein describe possible implementations, and that the operations and the steps may be rearranged or otherwise modified and that other implementations are possible. Furthermore, aspects from two or more of the methods may be combined.

The description set forth herein, in connection with the appended drawings, describes example configurations and does not represent all the examples that may be implemented or that are within the scope of the claims. The term “exemplary” used herein means “serving as an example, instance, or illustration,” and not “preferred” or “advantageous over other examples.” The detailed description includes specific details for the purpose of providing an understanding of the described techniques. These techniques, however, may be practiced without these specific details. In some instances, well-known structures and devices are shown in block diagram form in order to avoid obscuring the concepts of the described examples.

In the appended figures, similar components or features may have the same reference label. Further, various components of the same type may be distinguished by following the reference label by a dash and a second label that distinguishes among the similar components. If just the first reference label is used in the specification, the description is applicable to any one of the similar components having the same first reference label irrespective of the second reference label.

Information and signals described herein may be represented using any of a variety of different technologies and techniques. For example, data, instructions, commands, information, signals, bits, symbols, and chips that may be referenced throughout the description may be represented by voltages, currents, electromagnetic waves, magnetic fields or particles, optical fields or particles, or any combination thereof.

The various illustrative blocks and modules described in connection with the disclosure herein may be implemented or performed with a general-purpose processor, a DSP, an ASIC, an FPGA or other programmable logic device, discrete gate or transistor logic, discrete hardware components, or any combination thereof designed to perform the functions described herein. A general-purpose processor may be a microprocessor, but in the alternative, the processor may be any conventional processor, controller, microcontroller, or state machine. A processor may also be implemented as a combination of computing devices (e.g., a combination of a DSP and a microprocessor, multiple microprocessors, one or more microprocessors in conjunction with a DSP core, or any other such configuration).

The functions described herein may be implemented in hardware, software executed by a processor, firmware, or any combination thereof. If implemented in software executed by a processor, the functions may be stored on or transmitted over as one or more instructions or code on a computer-readable medium. Other examples and implementations are within the scope of the disclosure and appended claims. For example, due to the nature of software, functions described herein may be implemented using software executed by a processor, hardware, firmware, hardwiring, or combinations of any of these. Features implementing functions may also be physically located at various positions, including being distributed such that portions of functions are implemented at different physical venues. Also, as used herein, including in the claims, “or” as used in a list of items (for example, a list of items prefaced by a phrase such as “at least one of” or “one or more of”) indicates an inclusive list such that, for example, a list of at least one of A, B, or C means A or B or C or AB or AC or BC or ABC (i.e., A and B and C). Also, as used herein, the phrase “based on” shall not be construed as a reference to a closed set of conditions. For example, an exemplary step that is described as “based on condition A” may be based on both a condition A and a condition B without departing from the scope of the present disclosure. In other words, as used herein, the phrase “based on” shall be construed in the same manner as the phrase “based at least in part on.”

Computer-readable media includes both non-transitory computer storage media and communication media including any medium that facilitates transfer of a computer program from one place to another. A non-transitory storage medium may be any available medium that can be accessed by a general purpose or special purpose computer. By way of example, and not limitation, non-transitory computer-readable media can comprise RAM, ROM, electrically erasable programmable read-only memory (EEPROM), compact disk (CD) ROM or other optical disk storage, magnetic disk storage or other magnetic storage devices, or any other non-transitory medium that can be used to carry or store desired program code means in the form of instructions or data structures and that can be accessed by a general-purpose or special-purpose computer, or a general-purpose or special-purpose processor. Also, any connection is properly termed a computer-readable medium. For example, if the software is transmitted from a website, server, or other remote source using a coaxial cable, fiber optic cable, twisted pair, digital subscriber line (DSL), or wireless technologies such as infrared, radio, and microwave, then the coaxial cable, fiber optic cable, twisted pair, digital subscriber line (DSL), or wireless technologies such as infrared, radio, and microwave are included in the definition of medium. Disk and disc, as used herein, include CD, laser disc, optical disc, digital versatile disc (DVD), floppy disk and Blu-ray disc where disks usually reproduce data magnetically, while discs reproduce data optically with lasers. Combinations of the above are also included within the scope of computer-readable media.

The description herein is provided to enable a person skilled in the art to make or use the disclosure. Various modifications to the disclosure will be readily apparent to those skilled in the art, and the generic principles defined herein may be applied to other variations without departing from the scope of the disclosure. Thus, the disclosure is not limited to the examples and designs described herein, but is to be accorded the broadest scope consistent with the principles and novel features disclosed herein.

Any methods described in the claims or specification should not be interpreted to require the steps to be performed in a specific order unless stated otherwise. Also, the methods should be interpreted to provide support to perform the recited steps in any order unless stated otherwise.

Spatial or directional terms, such as “left,” “right,” “front,” “back,” and the like, relate to the subject matter as it is shown in the drawings. However, it is to be understood that the described subject matter may assume various alternative orientations and, accordingly, such terms are not to be considered as limiting.

Articles such as “the,” “a,” and “an” can connote the singular or plural. Also, the word “or” when used without a preceding “either” (or other similar language indicating that “or” is unequivocally meant to be exclusive—e.g., only one of x or y, etc.) shall be interpreted to be inclusive (e.g., “x or y” means one or both x or y).

The term “and/or” shall also be interpreted to be inclusive (e.g., “x and/or y” means one or both x or y). In situations where “and/or” or “or” are used as a conjunction for a group of three or more items, the group should be interpreted to include one item alone, all the items together, or any combination or number of the items.

The terms have, having, include, and including should be interpreted to be synonymous with the terms comprise and comprising. The use of these terms should also be understood as disclosing and providing support for narrower alternative embodiments where these terms are replaced by “consisting” or “consisting essentially of.”

Unless otherwise indicated, all numbers or expressions, such as those expressing dimensions, physical characteristics, and the like, used in the specification (other than the claims) are understood to be modified in all instances by the term “approximately.” At the very least, and not as an attempt to limit the application of the doctrine of equivalents to the claims, each numerical parameter recited in the specification or claims which is modified by the term “approximately” should be construed in light of the number of recited significant digits and by applying ordinary rounding techniques.

All disclosed ranges are to be understood to encompass and provide support for claims that recite any and all subranges or any and all individual values subsumed by each range. For example, a stated range of 1 to 10 should be considered to include and provide support for claims that recite any and all subranges or individual values that are between and/or inclusive of the minimum value of 1 and the maximum value of 10; that is, all subranges beginning with a minimum value of 1 or more and ending with a maximum value of 10 or less (e.g., 5.5 to 10, 2.34 to 3.56, and so forth) or any values from 1 to 10 (e.g., 3, 5.8, 9.9994, and so forth).

All disclosed numerical values are to be understood as being variable from 0-100% in either direction and thus provide support for claims that recite such values or any and all ranges or subranges that can be formed by such values. For example, a stated numerical value of 8 should be understood to vary from 0 to 16 (100% in either direction) and provide support for claims that recite the range itself (e.g., 0 to 16), any subrange within the range (e.g., 2 to 12.5) or any individual value within that range (e.g., 15.2).

The terms recited in the claims should be given their ordinary and customary meaning as determined by reference to relevant entries in widely used general dictionaries and/or relevant technical dictionaries, commonly understood meanings by those in the art, etc., with the understanding that the broadest meaning imparted by any one or combination of these sources should be given to the claim terms (e.g., two or more relevant dictionary entries should be combined to provide the broadest meaning of the combination of entries, etc.) subject only to the following exceptions: (a) if a term is used in a manner that is more expansive than its ordinary and customary meaning, the term should be given its ordinary and customary meaning plus the additional expansive meaning, or (b) if a term has been explicitly defined to have a different meaning by reciting the term followed by the phrase “as used in this document shall mean” or similar language (e.g., “this term means,” “this term is defined as,” “for the purposes of this disclosure this term shall mean,” etc.). References to specific examples, use of “i.e.,” use of the word “invention,” etc., are not meant to invoke exception (b) or otherwise restrict the scope of the recited claim terms. Other than situations where exception (b) applies, nothing contained in this document should be considered a disclaimer or disavowal of claim scope.

The subject matter recited in the claims is not coextensive with and should not be interpreted to be coextensive with any embodiment, feature, or combination of features described or illustrated in this document. This is true even if only a single embodiment of the feature or combination of features is illustrated and described in this document.

What is claimed:

1. A lip implant for implantation into lips of a patient, the lip implant includes:
an upper lip implant; and
a lower lip implant, wherein the upper lip implant and the lower lip implant are formed of an acrylate hydrogel.

2. The lip implant of claim 1, wherein the acrylate hydrogel comprises a polyacrylic hydrogel.

3. The lip implant of claim 1, wherein the acrylate hydrogel comprises a non-degradable acrylate hydrogel ink.

4. The lip implant of claim 1, wherein the acrylate hydrogel is derived from natural polymers.

5. The lip implant of claim 1, wherein the acrylate hydrogel is derived from synthetic polymers.

6. The lip implant of claim 1, wherein the acrylate hydrogel is derived from natural polymers and synthetic polymers.

7. The lip implant of claim 1, wherein the acrylate hydrogel is derived from acrylic acid.

8. The lip implant of claim 1, wherein the acrylate hydrogel is derived from acrylamide.

9. A method of designing lip implants, the method comprising:
scanning at least a lip of a patient using a scanner to generate anatomical data of the patient;
transmitting the anatomical data to a computing device;
creating a three dimensional model of the patient's lips using the computing device; and
designing the lip implant based at least partially on the three dimensional model.

10. The method of claim 9, wherein the three dimensional model comprises a computer aided design model.

11. The method of claim 9 further comprising analyzing the three dimensional model.

12. The method of claim 9 further comprising determining a horizontal lip position and fullness.

13. The method of claim 12, wherein determining a horizontal lip position and fullness comprises determining a horizontal lip position and fullness using a line method.

14. The method of claim 13, wherein the line method comprises drawing or rendering a line from a subnasale (Sn) of the patient through a labrale inferius (Li) of the patient to a pogonion (Pg) of the patient.

15. The method of claim 14, wherein the line method further comprises positioning an upper lip 3.5 millimeters anterior of the line and a lower lip 2.2 millimeters anterior of the line.

16. The method of claim 12, wherein determining a horizontal lip position and fullness comprises determining a horizontal lip position and fullness using a nasomental angle method.

17. The method of claim 16, wherein the nasomental angle method comprises drawing or rendering a first line along a bridge of a nose of the patient and a second line from a tip of the nose of the patient to a chin of the patient.

18. The method of claim 17, wherein the nasomental angle method further comprises positioning an upper lip 4 millimeters posterior of the second line and a lower lip 2 millimeters posterior of the second line.

19. The method of claim 18, wherein the first and second lines define an angle of about 1200 to about 132°.

20. A method of manufacturing lip implants, the method comprising:
depositing a first layer of acrylate hydrogel on a platform of an additive manufacturing system;
curing the first layer of acrylate hydrogel using a UV light of the additive manufacturing system;
depositing a second layer of acrylate hydrogel on the first layer; and
curing the second layer of acrylate hydrogel using the UV light of the additive manufacturing system.
